(19) United States
(12) Patent Application Publication
(10) Pub. No.: US 2024/0215892 A1
(43) Pub. Date: Jul. 4, 2024

(54) DEVICE FOR MONITORING BIOSIGNAL

(71) Applicant: HUINNO, CO., LTD., Seoul (KR)

(72) Inventors: Hwayoung SEO, Seoul (KR); Jung Hoon KIM, Hwaseong-si (KR); Youngsoo JANG, Bucheon-si (KR); Kyosik SIM, Gwangmyeong-si (KR); Eubin JEONG, Gunpo-si (KR); Joo Min KIM, Seoul (KR)

(21) Appl. No.: 18/606,955

(22) Filed: Mar. 15, 2024

Related U.S. Application Data
(63) Continuation of application No. PCT/KR2022/018051, filed on Nov. 16, 2022.

(30) Foreign Application Priority Data
Aug. 31, 2022 (KR) 10-2022-0110307

(51) Int. Cl. A61B 5/28; A61B 5/257; A61B 5/265

(52) U.S. Cl. CPC A61B 5/28; A61B 5/257; A61B 5/265

(57) ABSTRACT

A device for monitoring a biosignal includes a support where an electrode is formed; an attachment part formed on a lower surface of the support, and provided with a first hole formed in a position corresponding to the electrode; a first cover layer configured to cover a lower surface of the attachment part, and provided with a second hole formed in a position corresponding to the first hole; an attachment pad electrically connected to the electrode via the first hole and the second hole; and a second cover layer configured to cover a part of the first cover layer where the attachment pad is exposed.

### CROSS-REFERENCE TO RELATED APPLICATION(S)

This application is a Continuation of International Application No. PCT/KR2022/018051 filed on Nov. 16, 2022, which claims priority to Korean Patent Application No. 10-2022-0110307 filed on Aug. 31, 2022. The aforementioned applications are incorporated herein by reference in their entireties.

### TECHNICAL FIELD

The present invention relates to a device for monitoring a biosignal.

### RELATED ART

Due to recent rapid progress in science and technology, the quality of life of all mankind is being enhanced and medical environment has changed a great deal. Particularly, in recent years, wearable monitoring devices that can monitor electrocardiogram (ECG) signals during daily life without visiting a hospital have become widely available to the public.

Typically, conventional wearable monitoring devices employ off-the-shelf Ag/AgCl disposable electrodes, or are integrated with electrodes such that the electrodes are mechanically merged into a body with embedded circuitry for ECG signal monitoring.

Here, the wearable monitoring devices with integrated electrodes are aimed at long-term attachment, but there are two main problems. First, when the attachment location is corrected since it is incorrectly selected at the time of initial attachment, attachment strength of the attachment surface is deteriorated so that it is difficult to maintain attachment robustness. Second, when such a device is actually worn by a patient as prescribed, external factors such as skin condition of the patient or ingress of internal/external moisture may cause the attachment surface to become unavailable for continuous use before the end of the attachment period aimed for. In these cases, there is no way to restore or replace the deteriorated attachment strength, so that ECG data may not be obtained from the patient during the expected period, which can cause major problems in patient management.

### SUMMARY

One object of the present invention is to solve all the above-described problems in the prior art.

Another object of the invention is to provide a wearable monitoring device that may minimize deterioration of attachment strength of an attachment surface, while allowing an attachment location to be selected for clear biosignal measurement.

Yet another object of the invention is to provide a wearable monitoring device that allows only a part where an attachment surface is formed to be easily replaced, when an unintended deterioration of attachment strength of the attachment surface occurs while monitoring a biosignal.

The representative configuration of the invention to achieve the above objects is described below.

According to one aspect of the invention, there is provided a device for monitoring a biosignal, comprising: a support where an electrode is formed; an attachment part formed on a lower surface of the support, and provided with a first hole formed in a position corresponding to the electrode; a first cover layer configured to cover a lower surface of the attachment part, and provided with a second hole formed in a position corresponding to the first hole; an attachment pad electrically connected to the electrode via the first hole and the second hole; and a second cover layer configured to cover a part of the first cover layer where the attachment pad is exposed.

In addition, there are further provided other devices according to the technical idea of the invention.

According to the invention, it is possible to provide a wearable monitoring device that may minimize deterioration of attachment strength of an attachment surface, while allowing an attachment location to be selected for clear biosignal measurement.

According to the invention, it is possible to provide a wearable monitoring device that allows only a part where an attachment surface is formed to be easily replaced, when an unintended deterioration of attachment strength of the attachment surface occurs while monitoring a biosignal.

### BRIEF DESCRIPTION OF THE DRAWING

FIG. 1 is an exploded perspective view of a device according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the invention will be described in detail with reference to the accompanying drawings to enable those skilled in the art to easily implement the invention.

### Configuration of a Device

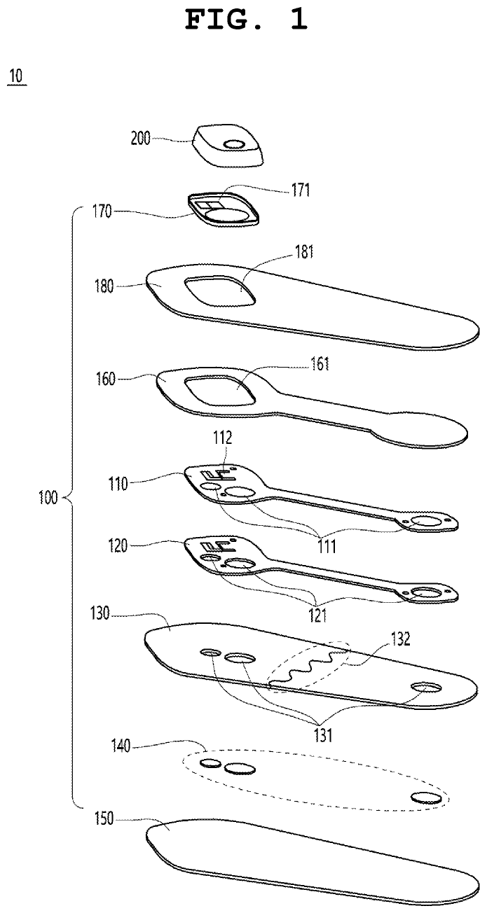

FIG. 1 is an exploded perspective view of a device 10 according to one embodiment of the invention.

Referring to FIG. 1, the device 10 according to one embodiment of the invention may include an electrode assembly 100 and a body housing 200. Here, the electrode assembly 100 according to one embodiment of the invention may include a support 110, an attachment part 120, a first cover layer 130, an attachment pad 140, a second cover layer 150, a waterproof layer 160, a receiving part 170, and a third cover layer 180. In the following, the detailed components of the electrode assembly 100 will be discussed, and then the body housing 200 will be discussed.

First, the support 110 according to one embodiment of the invention may comprise an electrode 111, a first connection member 112, and a trace (not shown) electrically connecting the electrode 111 and the first connection member 112. According to one embodiment of the invention, at least two electrodes 111 (preferably three electrodes including a reference electrode) may be formed in the support 110 at predetermined intervals. According to one embodiment of the invention, the electrode 111 may form a point of contact with a patient's body to measure a biosignal such as an electrocardiogram signal, and the biosignal measured from the electrode 111 may be transmitted to the first connection member 112 via the trace. Meanwhile, the support 110 according to one embodiment of the invention may be formed of a flexible material, such that the electrode 111 may easily form the point of contact with the patient's body.

Next, the attachment part 120 according to one embodiment of the invention may be formed on the lower surface of the support 110. According to one embodiment of the invention, the attachment part 120 may be formed of a material such as double-sided tape, such that one surface thereof may be attached to the lower surface of the support 110 and the other surface may be attached to the patient's body (but may be attached to the first cover layer 130 to be described later before the device 10 starts monitoring the patient's biosignal). That is, according to one embodiment of the invention, the other surface of the attachment part 120 may be an attachment surface that allows the device 10 to be attached to the patient's body.

Meanwhile, according to one embodiment of the invention, the attachment part 120 may have a first hole 121 formed in a position corresponding to the electrode 111 formed in the support 110. Here, the number of the first hole(s) 121 may correspond to the number of the electrode(s) 111 formed in the support 110, and the size or shape thereof may also correspond to the size or shape of the electrode(s) 111.

Next, the first cover layer 130 according to one embodiment of the invention may be configured to cover the lower surface of the attachment part 120, i.e., the attachment surface. That is, the first cover layer 130 according to one embodiment of the invention may be configured to cover the attachment surface before the device 10 starts monitoring the patient's biosignal (i.e., before the attachment surface is attached to the patient's body), thereby preventing attachment strength of the attachment surface from deteriorating. Meanwhile, according to one embodiment of the invention, no adhesive materials may be formed on both the surface of the first cover layer 130 in contact with the attachment part 120 (or the upper surface of the first cover layer 130) and the surface of the first cover layer 130 not in contact with the attachment part 120 (or the lower surface of the first cover layer 130).

Meanwhile, according to one embodiment of the invention, the first cover layer 130 may have a second hole 131 formed in a position corresponding to the first hole 121 formed in the attachment part 120 (or the electrode 111 formed in the support 110). Here, the number of the second hole(s) 131 may correspond to the number of the first hole(s) 121 formed in the attachment part 120 (or the number of the electrode(s) 111 formed in the support 110), and the size or shape thereof may also correspond to the size or shape of the first hole(s) 121 (or the size or shape of the electrode(s) 111).

Meanwhile, according to one embodiment of the invention, an incision 132 may be formed at one side of the first cover layer 130, such that the first cover layer 130 may be easily removed from the attachment part 120. According to one embodiment of the invention, the incision 132 may be formed across the first cover layer 130 to allow the first cover layer 130 to be separated into a plurality of pieces.

Next, the attachment pad 140 according to one embodiment of the invention may be electrically connected to the electrode 111 via (or by being inserted into) the first hole 121 and the second hole 131. That is, the attachment pad 140 according to one embodiment of the invention may assist the electrode 111 in measuring the patient's biosignal by forming a point of contact with the patient's body in place of the electrode 111. Here, the number of the attachment pad(s) 140 may correspond to the number of the electrode(s) 111, and the size or shape thereof may correspond to the size or shape of the electrode(s) 111 (or the size or shape of the first hole(s) 121 and the second hole(s) 131).

Next, the second cover layer 150 according to one embodiment of the invention may be configured to cover a part of the first cover layer 130 where the attachment pad 140 is exposed. According to one embodiment of the invention, the attachment pad 140 may be exposed to the outside via the second hole 131 formed in the first cover layer 130, and the second cover layer 150 may be configured to cover the second hole 131 to prevent the attachment pad 140 from being exposed to the outside before the device 10 starts monitoring the patient's biosignal (or before a location where the device 10 is attached to the patient's body is selected). Here, according to one embodiment of the invention, a release material may be formed on the surface of the second cover layer 150 in contact with the first cover layer 130 (i.e., the upper surface of the second cover layer 150), such that the second cover layer 150 may be attached to the attachment pad 140 before the device 10 starts monitoring the patient's biosignal, and may be removed from the attachment pad 140 just before the device 10 starts monitoring the patient's biosignal (or at a time when it is required to select the location where the device 10 is attached to the patient's body). Meanwhile, according to one embodiment of the invention, the surface of the second cover layer 150 not in contact with the first cover layer 130 (i.e., the lower surface of the second cover layer 150) serves as the lower surface of the device 10 before the device 10 starts monitoring the patient's biosignal, and no adhesive material may be formed thereon.

Next, the waterproof layer 160 according to one embodiment of the invention may be disposed on top of the support 110. Specifically, the waterproof layer 160 according to one embodiment of the invention may is formed of a waterproof material, and may be formed to cover the upper surface of the support 110 with a perimeter greater than the perimeter of the support 110. That is, the waterproof layer 160 according to one embodiment of the invention may block moisture, dust, and the like from entering the electrode 111 formed in the support 110.

Meanwhile, according to one embodiment of the invention, the waterproof layer 160 may have a third hole 161 formed at one side thereof, which allows the first connection member 112 to be exposed on the upper surface of the support 110. According to one embodiment of the invention, the size or shape of the third hole 161 may correspond to the size or shape of the receiving part 170 to be described below.

Next, the receiving part 170 according to one embodiment of the invention may be fixedly coupled to the support 110 via the third hole 161 formed in the waterproof layer 160. Further, the receiving part 170 according to one embodiment of the invention may have a fourth hole 171 formed in a position corresponding to the first connection member 112 (or a position corresponding to the third hole 161 formed in the waterproof layer 160), such that the first connection member 112 may be exposed on the upper surface of the support 110.

Meanwhile, the receiving part 170 according to one embodiment of the invention may have a fixing member (not shown) formed at one side thereof, which is configured to fix the body housing 200. For example, the fixing member may be formed along an inner perimeter of the receiving part 170, and may form a snag structure (or snap fit structure) together with a fixing member (not shown) formed along an outer perimeter of the body housing 200. According to one embodiment of the invention, the snag structure may allow the body housing 200 to be detachably received in (or coupled to) the receiving part 170.

Next, the third cover layer 180 according to one embodiment of the invention may be configured to cover the waterproof layer 160. According to one embodiment of the invention, the third cover layer 180 may be configured to cover the waterproof layer 160 to prevent the waterproof layer 160 from being exposed to the outside before the device 10 starts monitoring the patient's biosignal.

According to one embodiment of the invention, the third cover layer 180 may have a fifth hole 181 formed in a shape corresponding to the perimeter of the receiving part 170, such that the third cover layer 180 may be detached from the waterproof layer 160 and then removed as the receiving part 170 (or the receiving part 170 and the body housing 200 coupled to the receiving part 170) pass through the fifth hole 181.

Next, the body housing 200 according to one embodiment of the invention may include components for managing the biosignal measured through the electrode 111 formed in the support 110. According to one embodiment of the invention, the body housing 200 may have a second connection member (not shown) formed at one side thereof (e.g., on the lower surface of the body housing 200), which may be mounted on a printed circuit board assembly (PCBA) disposed within the body housing 200. According to one embodiment of the invention, the second connection member may be electrically connected to the first connection member 112 via the third hole 161 and the fourth hole 171, and may receive the biosignal measured through the electrode 111 from the first connection member 112. Meanwhile, according to one embodiment of the invention, in addition to the second connection member, a memory for recording the biosignal, a processor for processing the biosignal (e.g., estimating arrhythmia from the biosignal if the biosignal is an electrocardiogram signal), a communication module for transmitting the biosignal to the outside (via wired or wireless transmission), and the like may be further mounted on the PCBA disposed within the body housing 200.

Meanwhile, the body housing 200 according to one embodiment of the invention may have a fixing member (not shown) formed at one side thereof as described above, which may form a snag structure (or snap fit structure) together with the fixing member formed in the receiving part 170, such that the body housing 200 may be detachably coupled to the receiving part 170 as the body housing 200 is received in the receiving part 170. According to one embodiment of the invention, since the body housing 200 is detachably coupled to the receiving part 170, the electrode assembly 100 may be easily replaced with another electrode assembly 100 by detaching the body housing 200 from the receiving part 170 when an unintended deterioration of attachment strength of the attachment surface occurs while the device 10 monitors the biosignal. This allows biosignal data stored in the memory within the body housing 200 to be preserved, while allowing the biosignal measurement to continue as long as the monitoring is required.

### How to Attach the Device to a User'S Body

In the foregoing, the components included in the device 10 have been discussed in detail. In the following, how to attach the device 10 to a user's body will be discussed in detail, focusing on the first cover layer 130 and the second cover layer 150 among the components included in the device 10.

According to one embodiment of the invention, the device 10 may be delivered to a patient in a state in which the electrode assembly 100 and the body housing 200 are coupled and the respective components of the electrode assembly 100 are coupled.

First, according to one embodiment of the invention, the second cover layer 150 disposed at the lowermost part of the device 10 is removed from the device 10. According to one embodiment of the invention, in a state in which the second cover layer 150 is removed from the device 10, only the first cover layer 130 and the attachment pad 140 are exposed at the lower part of the device 10.

Here, since no adhesive material is formed on the lower surface of the first cover layer 130, the device 10 will not be attached to the patient's body even if the device 10 is in contact with the patient's body in this state.

Next, according to one embodiment of the invention, in a state in which the first cover layer 130 and the attachment pad 140 are exposed at the lower part of the device 10, the device 10 is placed at various locations on the patient's body to observe the patient's biosignal, and the location where the biosignal is most clearly measured is selected. Further, the selected location is indicated by a marker.

Next, according to one embodiment of the invention, the device 10 is removed from the patient's body and the first cover layer 130 is removed from the device 10. According to one embodiment of the invention, in a state in which the first cover layer 130 is removed from the device 10, only the attachment part 120 and the attachment pad 140 may be exposed at the lower part of the device 10.

Next, according to one embodiment of the invention, in a state in which the attachment part 120 and the attachment pad 140 are exposed at the lower part of the device 10, the lower surface of the attachment part 120, i.e., the attachment surface is attached to the location indicated by the marker.

Lastly, according to one embodiment of the invention, in a state in which the attachment surface is attached to the patient's body (i.e., the location indicated by the marker), the third cover layer 180 is removed from the device 10.

Thus, the present invention may minimize deterioration of attachment strength of the attachment surface that may occur during the process of selecting an attachment location for the device 10, and select an optimal location for clear biosignal measurement on the patient's body.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A device for monitoring a biosignal, comprising:
a support where an electrode is formed;
an attachment part formed on a lower surface of the support, and provided with a first hole formed in a position corresponding to the electrode;
a first cover layer configured to cover a lower surface of the attachment part, and provided with a second hole formed in a position corresponding to the first hole;
an attachment pad electrically connected to the electrode via the first hole and the second hole; and
a second cover layer configured to cover a part of the first cover layer where the attachment pad is exposed.

2. The device of claim 1, further comprising:
a waterproof layer formed to cover an upper surface of the support with a perimeter greater than a perimeter of the support, and provided with a third hole formed at one side thereof, wherein the third hole allows a first connection member electrically connected to the electrode to be exposed on the upper surface of the support.

3. The device of claim 2, further comprising:
a receiving part coupled to the support via the third hole, and provided with a fourth hole formed in a position corresponding to the first connection member, and a body housing fixing member formed at one side thereof.

4. The device of claim 3, further comprising:
a third cover layer configured to cover the waterproof layer, and provided with a fifth hole formed in a shape corresponding to a perimeter of the receiving part.

5. The device of claim 3, further comprising:
a body housing detachably coupled to the receiving part, and provided with a second connection member formed at one side thereof, wherein the second connection member is electrically connected to the first connection member via the third hole and the fourth hole.
